(19) United States
(12) Patent Application Publication
(10) Pub. No.: US 2015/0191409 A1
(43) Pub. Date: Jul. 9, 2015

(54) Process

(71) Applicant: NATURALLY SCIENTIFIC TECHNOLOGIES LIMITED, Buckinghamshire (GB)

(72) Inventor: Glenn RICHARDS, Rochester, NY (US)

(73) Assignee: NATURALLY SCIENTIFIC TECHNOLOGIES LIMITED, Buckinghamshire (GB)

(21) Appl. No.: 14/148,974

(22) Filed: Jan. 7, 2014

(51) Int. Cl. C07C 51/00; C07C 67/03; B01F 5/00; C07C 29/09

(52) U.S. Cl. CPC C07C 51/00; C07C 29/095; C07C 67/03; B01F 5/00; B01F 2215/0067

(57) ABSTRACT

The present invention relates to a process, which is particularly suitable for the production of sebacic acid and/or 2-octanol, and also for the production of biodiesel, wherein the process is a process for the reaction of a fatty acid ester (such as castor oil, or canola or rapeseed oil) and/or a fatty acid (such as riconleic acid) with an inorganic base (such as sodium hydroxide), the process comprising the following steps: (i) providing a pressurised and heated stream of fatty acid ester and/or fatty acid; (ii) providing an inorganic base; (iii) combining the stream obtained from step (i) with the inorganic base obtained in step (ii), in the presence of water, in a reaction vessel, by injection, thereby to produce reaction mixture in the form of a pressurised and heated stream; (iv) optionally maintaining the stream obtained in step (iii) at a selected temperature and pressure; and (v) thereby obtaining a reaction product.

### BACKGROUND

### Field of the Invention

The invention relates to a process for the reaction of a fatty acid ester and/or a fatty acid with a reactant, such as an inorganic base. In two particular embodiments, the invention relates to improved processes for the production of sebacic acid from castor oil, and for the production and biodiesel from rapeseed oil or other oils.

### INTRODUCTION

The listing or discussion of an apparently prior-published disclosure in this specification should not necessarily be taken as an acknowledgement that the document is part of the state of the art or is common general knowledge.

The conversion of oils, such as castor oil, into commercially valuable products is well known. In particular, the reaction to cleave castor oil in a strongly basic solution under heat to produce sebacic acid and 2-octanol is well known in the art.

U.S. Pat. No. 2,182,056 discloses the preparation of sebacic acid using castor oil as the starting reagent. Sodium hydroxide is added to the castor oil in the presence of a catalyst in an alkali fusion reaction to give products such as sebacic acid and 2-octanol.

The major problem with the existing production of sebacic acid is that the transition metal catalysts which are typically used in the process are oxides and chlorides of lead, bismuth, cadmium and titanium which are partially soluble in water. In particular, U.S. Pat. No. 3,031,482 discloses using lead, bismuth, thallium as catalytically active metal compounds in the conversion of ricinoleates into sebacic acid. These heavy metals are toxic and their release into water supplies is highly undesirable. It would therefore be advantageous to find a process which does not require the use of such catalysts. However, prior to the present invention, it has been found to be very difficult to moderate the reaction without using these catalysts.

Furthermore, the processing and reactions involving viscous oils present technical difficulties. For example, castor oil has a very high viscosity of approximately 600 centipoise (cP) when measured at 20° C. and 1 atm. When undergoing processing, very viscous fluids, such as castor oil, tend to form plugs as they pass through pipes, and are also resistant to mixing in reaction vessels, leading to uneven distribution of reaction components and uneven temperature distribution within the reaction vessel. Previously, the problems caused by the high viscosity of such oils have been addressed by the addition of diluents, or non-reactive processing aids, in order to lower the viscosity. The diluents are also added to prevent the oils from burning when they are subjected to high temperatures. A number of attempts have been made to moderate the reaction using non-reactive processing aids such as mineral oil, polyethylene glycol (PEG), waxes and paraffin.

For example, U.S. Pat. No. 6,392,074 discloses using low melting aliphatic organic isocarboxylic acids containing 5 to 13 carbon atoms as a diluent to address the problem of excessive viscosity thickening in the cleavage reaction of castor oil.

Although the addition of such non-reactive diluents aids with the uniformity of the reaction and reduces thermal degradation, they do not improve upon the yield of the reaction products. A further problem which arises from using non-reactive diluents is that further processing steps will then be required in order to further separate the diluents from the products which result from the processed oils, resulting in a more complex production process and higher production costs.

Without the use of diluents and processing aids, the viscous reaction mixture is very difficult to stir and maintain at a constant temperature, and thus hot spots tend to form within the mixture itself. Similar problems not only occur when using viscous oils such as castor oil but also, for example, during the synthesis of biodiesel when reacting oils, typically vegetable oils such as rapeseed oil, with reactants such as methanol and sodium hydroxide.

Castor oil, in particular, has a very high viscosity until it is heated over 200° C. Therefore, when synthesising sebacic acid, high powered mixers and agitators have normally been required to mix the castor oil and sodium hydroxide. Furthermore, high pressure sealed reactor vessels have been used which are not only expensive, but are notoriously difficult to maintain motor shaft seals under the required operating conditions. In these existing systems, it has been difficult to heat the reaction mixture uniformly.

Reaction times for sebacic acid from castor oil have varied depending on temperature, mixing conditions, the initial viscosity of the castor oil, the catalyst used, weight ratio of catalyst to oil and various other factors.

It is also known in the art to perform reactive distillation of sebacic acid and 2-octanol from castor oil using microwave irradiation. Nezihe Azcan et al., 2008, Ind. Eng. Chem. Res., 47, 1774-1778 discloses a reaction which was performed in a microwave synthesis unit at 240° C. to give a 20 minute reaction time. By using microwave as the heat source, the reaction time was reduced from the several hours it typically takes, to approximately 20 minutes. However, on a commercial scale this procedure is not viable because it is very expensive to make large enough pressure vessels to hold the castor oil with the required catalysts as well as keeping the microwave reactors under a suitable pressure.

There have also been attempts to use sonic reactors in which the entire vessel has sonic waves passing through the liquid media under pressure. Although these very expensive reactors do a good job mixing viscous materials, they are not suitable for large scale commercial production. Scaling up a sonic reactor that is over 2 meters high becomes difficult in terms of mounting and vibrating the large mass of metal and fluids. Furthermore, the acoustic waves can damage tissue in the body making the scale up dangerous to operate.

Another problem faced by the manufacturer of sebacic acid is that excess sodium hydroxide can accumulate in the heavy metal catalysts. In order to maintain productivity, the catalyst mixture is regularly refreshed by dumping unreacted material into the waste stream, ultimately resulting in serious environmental pollution.

It is therefore desirable to have a new process which uses no catalyst (thereby avoiding the environmental impact), has highly uniform reaction temperatures throughout the reaction process and minimizes capex.

The present inventors have developed new a process which comprises several new and innovative steps and process features, as will be described in more detail below, and which surprisingly address the foregoing disadvantages and problems in the art. The combination of these new and innovative steps and process features provides processes for the highly efficient reaction of fatty acid esters and/or fatty acids or derivatives thereof. For example, the process developments of the present invention result in improved methods for the production of sebacic acid; and biodiesel.

Accordingly, it is an object of the present invention to provide a method to convert castor oil to sebacic acid without metal catalysts that might include lead and other toxic metals, and to provide a method to convert vegetable oils to biodiesel at high efficiencies with faster reaction times.

It is also an object of the present invention to provide process equipment which facilitates a novel means of holding temperatures uniform throughout the reaction fluid mass without external mixing motors. As described below, this object can be addressed with novel combinations of heat exchangers, sonic nozzles and/or static mixers.

### SUMMARY

Accordingly, the present invention provides a process for the reaction of a fatty acid ester and/or a fatty acid with an inorganic base, the process comprising the following steps:
(i) providing a pressurised and heated stream of fatty acid ester and/or fatty acid;
(ii) providing an inorganic base;
(iii) combining the stream obtained from step (i) with the inorganic base obtained in step (ii), in the presence of water, in a reaction vessel, by injection, thereby to produce reaction mixture in the form of a pressurised and heated stream;
(iv) optionally maintaining the stream obtained in step (iii) at a selected temperature and pressure; and
(v) thereby obtaining a reaction product.

The process may be conducted in the absence of transition metal catalyst. The process may be conducted in the absence of non-reactive diluents or other non-reactive processing aids which reduce the viscosity of the reaction mixture, including mineral oil, polyethylene glycol (PEG), waxes and/or paraffin.

According to one embodiment, the pressurised and heated stream of fatty acid ester and/or fatty acid may be provided, in step (i), in a continuous unidirectional flow along the lumen of one or more tubes or other conduits, and the, or each, lumen operates as the reaction vessel in step (iii), and preferably under conditions which cause turbulent flow and/or a Reynolds number of greater than 2000, such at least 2040, 2100, 2500, 3000, 4000, 5000 or greater.

According to a further embodiment, step (iii) of the process may comprise the injection of the inorganic base into the reaction vessel at a speed which is high enough to prevent laminar flow of the injected inorganic base, and preferably using at least one sonic nozzle.

According to a further embodiment, step (i) of the process may comprise pumping the fatty acid ester and/or fatty acid into the lumen of a tube or other conduit to create a pressurised continuous unidirectional flow of fatty acid through the lumen, and preheating the pressurised continuous unidirectional flow of fatty acid ester and/or fatty acid in the presence of water, for example by steam injection.

According to a further embodiment, the pressurised and heated stream of fatty acid ester and/or fatty acid is mixed, during step (i), during step (iii), and/or after step (iii), using one or more static mixers. For example, the static mixers used during step (i), during step (iii), and/or after step (iii), may achieve a Reynolds numbers of at least about 2040 to 10,000, preferably at a least about 2500, 3000, 4000, 5000 or more.

According to a further embodiment, the pressurised and heated stream provided in step (i), during step (iii), and/or after step (iii) of the process may be a continuous unidirectional flow along the lumen of one or more tubes or other conduits, and the temperature of the continuously flowing stream may vary across the cross-section of the lumen of the tube or other conduit by no more than 10° C., 5° C., 2° C., 1° C., 0.5° C., 0.1° C., or less than 0.1° C., at any given point along the length of continuous flow through the lumen.

According to a further embodiment, the reaction vessel used in step (iii) of the process may be a heat exchanger, which comprises one or more hollow tubes or other conduits, and according to this embodiment, the reaction occurs in a continuous flow within the lumen of the one or more hollow tubes or other conduits. Preferably, according to this embodiment, in step (iv) of the process, the stream may be maintained at a selected temperature and pressure during the continuous flow within the lumen of the one or more hollow tubes or other conduits. Further preferably, according to this embodiment, the path length of the continuous flow within the lumen of the one or more hollow tubes or other conduits of the heat exchanger may be selected to permit the desired level of product formation. Yet further preferably, the path length may be extended by continuously recycling the flow around a circuit within the heat exchanger until the product forms.

According to a further embodiment, the stream in steps (i) and/or (iii) of the process may be pressurized by using at least one high pressure high temperature pump, such as a high pressure high temperature pump which operates at a pressure of 10 to 50 bar (such as, at least 15, 20, 25, 30, 35 or 40 bar) and a temperature of 100 to 400° C. (such as at least 150, 200, 250, 300, or 350° C.).

According to a further embodiment, step (i) of the process may comprise providing a pressurised and heated stream of fatty acid ester, wherein at 20° C. and 1 atm the fatty acid ester has a viscosity of greater than about 200 cP, about 300 cP, about 400 cP, about 500 cP, or about 600 cP

According to a further embodiment, the fatty acid ester used in the process may comprise castor oil and/or the fatty acid comprises ricinoleic acid, and preferably according to this embodiment the reaction product comprises sebacic acid and/or 2-octanol. Further, preferably, according to this embodiment, step (i) of the process may comprise pumping castor oil at a pressure of about 10 to 50 bar (such as, at least 15, 20, 25, 30, 35 or 40 bar) and at a temperature from about 200° C. to about 320° C. (such as at least 220, 240, 260, 280, or 300° C.), preferably from about 300° C. to about 320° C. Yet further preferably, according to this embodiment, step (i) of the process may comprise steam injection into the stream of pumped castor oil. It may be preferred, according to this embodiment that the inorganic base is sodium hydroxide. In accordance with this embodiment, the inorganic base may be injected into the heated and pressurised stream of castor oil and/or ricinoleic acid using one or more sonic nozzles or, in an alternative, the inorganic base may be blended with castor oil and/or ricinoleic acid and the blend is passed through one or more sonic nozzles. In a further preference, in accordance with this embodiment, the castor oil and/or ricinoleic acid may be reacted with inorganic base, in the presence of water, at about 320° C. under pressure of about 10-15 bar. The castor oil and/or ricinoleic acid may preferably be reacted with inorganic base, in the presence of water, in a continuous unidirectionally flowing stream, wherein the stream passes over and is mixed by one or more in-line static mixers. The castor oil and/or ricinoleic acid may preferably be reacted with inorganic base, in the presence of water, in the lumen of one or more tubes or conduits, and preferably the one or more tubes or conduits form a heat exchanger.

The present invention also provides for the use of one or more static mixers, optionally made of stainless steel, to mix a heated and pressurised stream of castor oil, wherein the stream passes through the lumen of a tube or other conduit, and the one or more static mixers are positioned within the lumen. Preferably, this use is for performing any process of the present invention as defined above.

The present invention also provides for the use of one or more sonic nozzles to introduce inorganic base into a reaction vessel, thereby combining the inorganic base with fatty acid ester and/or fatty acid. Preferably, this use is for performing any process of the present invention as defined above.

The present invention also provides for the use of one or more sonic nozzles to introduce steam into a reaction vessel, wherein the reaction vessel comprises a reaction mixture of fatty acid ester and/or fatty acid with inorganic base, wherein the introduction of steam using the one or more sonic nozzles causes the heating and mixing of the reaction mixture. Preferably, this use is for performing any process of the present invention as defined above.

The present invention also provides for the use of a heat exchanger as a reaction vessel for the reaction of fatty acid ester and/or fatty acid, with organic base, in the presence of water, optionally wherein the heat exchanger is a shell in tube heat exchanger and the use comprises the provision of super-heated steam in the shell. Preferably, this use is for performing any process of the present invention as defined above.

In an alternative embodiment of the process of the present invention, the process step (iii) of the process may comprise combining the stream obtained from step (i) of claim 1 with the inorganic base obtained in step (ii) of claim 1, in the presence of water and a C₁-C₃ mono-alcohol (such as methanol, ethanol or propanol), in a reaction vessel, by injection, thereby to produce reaction mixture in the form of a pressurised and heated stream. In accordance with this embodiment, step (i) of the process may comprise providing a pressurised and heated stream of fatty acid ester, wherein at 25° C. and 1 atm the fatty acid ester has a viscosity of less than about 200 cP, about 150 cP, or about 100 cP. For example, the fatty acid ester employed in this embodiment may comprise canola or rapeseed oil, and/or preferably the reaction product may comprise biodiesel and/or glycerol. Optionally, the biodiesel may be, or comprise, or consist essentially of, or consist of, one or more compounds selected from fatty acid methyl ester, fatty acid ethyl ester, and/or fatty acid propyl ester. Optionally, the fatty acid ester comprises canola or rapeseed oil, and step (i) of the process comprises pumping canola or rapeseed oil at a pressure of about 5 bar 10, and at a temperature from about 50° C. (such as at least 55° C., 60° C., 65° C., or 70° C.) to about 80° C. (such as up to 75° C., 70° C., 65° C., or 60° C.). In accordance with this alternative embodiment, it may be preferred that step (i) of the process comprises steam injection into the stream of pumped fatty acid ester. It may further be preferred that the inorganic base is sodium hydroxide or potassium hydroxide. In a further option, the inorganic base may be injected into the heated and pressurised stream of fatty acid ester and/or fatty acid using one or more sonic nozzles or, in an alternative option, the inorganic base may be blended with fatty acid ester and/or fatty acid and the blend is passed through one or more sonic nozzles. In one preferred option, the fatty acid ester is canola or rapeseed oil and it is reacted with the inorganic base, in the presence of water and C₁-C₃ mono-alcohol (such as methanol or ethanol), at about 50-60° C. under pressure of about 3-5 bar. Optionally, according to this alternative embodiment, the fatty acid ester and/or fatty acid may be reacted with inorganic base, in the presence of water and C₁-C₃ mono-alcohol (such as methanol or ethanol), in a continuous unidirectionally flowing stream, wherein the stream passes over and is mixed by one or more in-line static mixers. Further, optionally, according to this alternative embodiment, the fatty acid ester and/or fatty acid may be reacted with inorganic base, in the presence of water and C₁-C₃ mono-alcohol (such as methanol or ethanol), in the lumen of one or more tubes or conduits, and preferably the one or more tubes or conduits form a heat exchanger.

The present invention also provides for the use of one or more static mixers, optionally made of stainless steel, to mix a heated and pressurised stream of fatty acid ester, particularly canola or rapeseed oil, wherein the stream passes through the lumen of a tube or other conduit, and the one or more static mixers are positioned within the lumen. Preferably, this use is for performing any process as defined by the alternative embodiment as described above.

The present invention also provides for the use of one or more sonic nozzles to introduce inorganic base and/or C₁-C₃ mono-alcohol into a reaction vessel, thereby combining inorganic base, C₁-C₃ mono-alcohol and fatty acid ester and/or fatty acid, preferably wherein the use is for the production of biodiesel. Preferably, this use is for performing any process as defined by the alternative embodiment as described above.

The present invention also provides for the use of one or more sonic nozzles to introduce steam into a reaction vessel, wherein the reaction vessel comprises a reaction mixture of fatty acid ester and/or fatty acid, inorganic base, C₁-C₃ mono-alcohol, and wherein the introduction of steam using the one or more sonic nozzles causes the heating and mixing of the reaction mixture, preferably wherein the use is for the production of biodiesel. Preferably, this use is for performing a process as defined by the alternative embodiment as described above.

The present invention also provides for the use of a heat exchanger as a reaction vessel for the reaction of fatty acid ester and/or fatty acid, with organic base and C₁-C₃ mono-alcohol, in the presence of water, optionally wherein the heat exchanger is a shell in tube heat exchanger and the use comprises the provision of super-heated steam in the shell, and preferably wherein the use is for the production of biodiesel. Preferably, this use is for performing a process as defined by the alternative embodiment as described above.

It may be particularly preferred that any process of the present invention, as described above, provides reaction products following step (v) which comprise no, or substantially no (such as less than 200, 100, 65, 50, 25, 10, 5, 4, 3, 2, or 1 ppm or, expressed in the alternative, less than 5%, 4%, 3%, 2%, 1%, 0.5%, 0.4%, 0.3%, 0.2%, 0.1%, 0.05%, 0.01%, 0.005%, or 0.001% by weight) transition metal catalyst, and preferably wherein the reaction products comprise sebacic acid and/or 2-octanol.

It may be particularly preferred that any process of the present invention, as described above, provides reaction products produced following step (v) which comprise no, or substantially no (such as less than 10%, 5%, 4%, 3%, 2%, 1%, 0.5%, or 0.1% by weight), non-reactive diluents or other non-reactive processing aids which reduce the viscosity of the reaction mixture, including mineral oil, polyethylene glycol (PEG), waxes and/or paraffin, and preferably wherein the reaction products comprise sebacic acid and/or 2-octanol.

Optionally, in accordance with any process of the present invention, following step (v), one or more of the reaction products is/are collected and/or purified, preferably wherein the collected and/or purified products are selected from the group consisting of sebacic acid, 2-octanol, biodiesel, and glycerol. Collection and/or purification may comprise feeding the reaction product collected from step (v) into a settling tank. Additionally, or alternatively, collection and/or purification may comprise a distillation step. Additionally, or alternatively, collection and/or purification may comprises the capture of unreacted products, such as unreacted inorganic base, unreacted fatty acid ester, unreacted fatty acid, unreacted C₁-C₃ mono-alcohol and/or water, and optionally the process further comprises feeding the one or more captured unreacted products back into the reaction process. For example, unreacted inorganic base, such as sodium hydroxide, may be captured using an ion exchanger. Additionally, or alternatively, collection and/or purification may comprise the removal of darkened FAE, for example, by nanofiltration or activated charcoal. Accordingly, the process of the present invention may be a process for obtaining a purified sebacic acid product, or a process for obtaining a purified 2-octanol product, or a process for obtaining a purified biodiesel product.

The process of the present invention may run the reaction as a continuous process, or a batch process.

In the embodiment that the process of the present invention is a process for obtaining a purified sebacic acid product, then the process may further comprise reacting or formulating the purified sebacic acid to form one or more additional products. For example, the one or more additional products may be selected from the group consisting of chemical compounds, such as esters and polymer resins, including nylon 6/10, which in turn may be fabricated into further products (such as toothbrush bristles, fishing lines, and paper machine felts), or polymers comprising sebacic acid such as polyester resins, coatings, and adhesives, or the products of reacting sebacic acid with various alcohols to produce sebacate esters, which are useful as plasticizers (which soften stiff plastics and resins) for example in producing polyvinyl chloride (PVC) films to provide low temperature flexibility and freedom from cracking, or by formulation of sebacic acid in products such as antifreeze coolants, corrosion inhibitors in cutting and metal-working fluids, and in other formulated products such as coatings, and lubricants.

In the embodiment that the process of the present invention is a process for obtaining a purified 2-octanol product, then the process may further comprise reacting or formulating the purified 2-octanol to form one or more additional products.

In the embodiment that the process of the present invention is a process for obtaining a purified biodiesel product, then the process may further comprise reacting or formulating the purified biodiesel to form one or more additional products.

The present invention also provides one or more products obtainable from any of the above-defined processes or uses of the present invention.

The present invention also provides an apparatus for use in any of the above-defined processes or uses of the present invention, the apparatus comprising:
a reaction vessel formed from the lumen of at least one hollow tube or other conduit, to permit continuous unidirectional flow of a reaction mixture through the lumen
at least one pump configured to pump a reaction mixture comprising fatty acid ester and/or fatty acid through the lumen; and
at least one static mixer positioned within the lumen, configured to mix the reaction mixture as it passes through the lumen and over the static mixer.

Optionally, the pump may be connected to a source of fatty acid ester and/or fatty acid, to permit the pumping the fatty acid ester and/or fatty acid through the lumen.

Further optionally, the apparatus may further comprise at least one steam or sonic nozzle for injecting a pressurised stream of inorganic base and/or C₁-C₃ mono-alcohol into the reaction vessel, optionally connected to a source of inorganic base and/or to a source of C₁-C₃ mono-alcohol.

Further optionally, the apparatus may further comprise one or more steam injection ports, for injecting steam into the lumen, and thereby heating and mixing the stream of fatty acid ester and/or fatty acid, or reaction mixture comprising fatty acid ester and/or fatty acid, as it passes through the lumen, optionally connected to a source of pressurised steam.

Further optionally, the reaction vessel of the apparatus may comprise a recirculation loop, to permit the diversion of reaction mixture from the lumen of the at least one hollow tube or other conduit and cause the re-entry of the diverted reaction mixture to the lumen at a point upstream of the position diversion point.

Further optionally, the apparatus may further comprise a preheating and pumping apparatus for pressurising and heating the fatty acid ester and/or fatty acid before it is passed into the reaction vessel, optionally comprising a pump, one or more steam injection ports, and/or one or more static mixers.

Further optionally, the reaction vessel of the apparatus of the present invention may be a heat exchanger.

Further optionally the apparatus may further comprise a purification and/or separation means to permit the purification and/or separation of one or more reaction products and/or one or more unreacted components. The purification and/or separation means may, for example, be adapted to permit the separation of unreacted fatty acid ester, unreacted fatty acid, unreacted inorganic base, unreacted C₁-C₃ mono-alcohol and/or water from the reaction product. Optionally, the purification and/or separation means may be adapted to separate sebacic acid and/or 2-octanol, either together or more preferably separately, from the initial reaction product. In an alternative option, the purification and/or separation means may be adapted to separate biodiesel and/or glycerol, either together or more preferably separately, from the initial reaction product. For example, the purification and/or separation means may comprise means selected from means for distillation, a settling tank, an ion exchanger, and/or a nanofilter or activated carbon filter.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIG. 1 is a schematic diagram of a preferred process of preparing sebacic acid and octanol-2 from castor oil.

FIG. 2 is a schematic diagram of the process of a preferred preparing biodiesel from rapeseed oil.

### DETAILED DESCRIPTION

As used herein, the term “fatty acid ester”, which is also abbreviated herein to FAE, refers to a substance that comprises, consists essentially of, or consists of, a type of ester that results from the combination of a fatty acid with an alcohol.

A substance that consists essentially of a FAE refers to a substance that may comprise other components, but which do not materially affect the ability of the FAE to be reacted in the process of the present invention (for example, do not reduce the overall yield by more than 10%, 5%_{,} 4%_{,} 3%, 2%, 1% or less). For example, a substance that consists essentially of a FAE may comprise at least 50%, 60%, 70%, 80%, 90%, 95%, 96%, 97%, 98%, 99%, 99.5%, 99.9% or 100% by weight fatty acid ester, the remainder being some other component or components. A common additional component provided with FAE may be water. Another common additional component, in particular in the case that the FAE is a plant or vegetable oil or fat, may be plant-derived debris, such as cell wall components, cellular proteins, nucleic acids, and the like. Others components found in vegetable oils include residual solvents such as hexane used for oil extraction and/or carbohydrates found in the seed prior to their passing through the expeller press. In one preferred embodiment, in the case of plant or vegetable oils and fats, the oil or fat will have been filtered or otherwise treated to remove particular matter, such as particles and other impurities greater than 100 μm, 50 μm, 20 μm, 10 μm, or 5 μm in size, in at least one dimension.

It may be particularly preferred that a substance which comprises or consists essentially of FAE contains no, or substantially no (for example, no more than 10%, 5%, 4%, 3%, 2%, 1% by weight), viscosity-lowering diluents or non-reactive processing aids, especially any one or more of mineral oil, polyethylene glycol (PEG), waxes and paraffin.

It may be particularly preferred that a substance which comprises or consists essentially of FAE contains no, or substantially no, transition metal catalysts, such as oxides and/or chlorides of one or more (including all) of lead, bismuth, cadmium and titanium.

Typically, although not necessarily, the alcohol component of a fatty acid ester will be glycerol.

FAE may comprise one or more, such as two or three or more fatty acids joined to an alcohol by esterification. These may be the same, or different, fatty acids. In the instance that the alcohol is glycerol, then the fatty acid esters may be monoglycerides, diglycerides, or triglycerides, all of which are components of vegetable fats and oils.

Accordingly, in one embodiment, a preferred FAE for use in the present invention may be a vegetable oil or fat. A vegetable oil or fat is derived from a plant or vegetable source, such as from agriculturally grown plant material, or a part thereof (most commonly the seed), or plant material that has been cultured.

For example, vegetable fats and oils can be recovered from plant material, including seeds, by mechanical extraction (such as, crushing or pressing), including expeller-pressing extraction, screw pressing, ram pressing, Ghani (powered mortar and pestle) extraction, and oil seed presses. Another alternative is solvent extraction, which is commonly done by chemical extraction, using solvent extracts, which usually produces higher yields and is quicker and less expensive than mechanical extraction. The most common solvent is hexane, although other solvents may be used such as a combination of isopropanol and hexane or hexane and ethanol. Supercritical carbon dioxide can be used as a non-toxic alternative to other solvents.

Of particular note for the purposes of the present invention is the methodology for the growth of plant cells in suspension culture and the production of vegetable oils therefrom, as described in WO 2009/133351 and WO 2012/160360, the contents of each of which are incorporated herein by reference. Vegetable oils produced according to the methods described in WO 2009/133351 and WO 2012/160360 are typically more pure (for example, having reduced plant debris and/or no solvent component) and/or more homogenous (in the sense that there is reduced variation in the fatty acid components) compared to agriculturally-derived vegetable oils that have been obtained by mechanical or solvent extraction. Accordingly, vegetable oil processed according to the present invention may preferably be vegetable oil derived from a process described in WO 2009/133351 and/or WO 2012/160360, and so a process according to the present invention may also comprise a first stage of manufacturing a vegetable oil by a process as described in WO 2009/133351 and/or WO 2012/160360, and then a second stage of reacting the vegetable oil by a process in accordance with the present invention.

Oils from other plant and algal cell cultures known in the art may also be used in the present invention.

One advantage of the present invention is that, in at least one embodiment, it is not necessary to remove water from the FAE before its use in a downstream process of the invention. Water may, for example, be present in FAEs derived from plant and algal cell cultures.

Of particular interest for use in the practice of the present invention is high viscosity FAE, including high viscosity vegetable oils, especially castor oil which has a viscosity of about 600 cP. Other high viscosity FAE suitable for use in the present invention can include oils having a viscosity of at least about 200 cP, at least about 300 cP, at least about 400 cP, at least about 450 cP, at least about 500 cP, at least about 550 cP, or at least about 600 cP. The foregoing viscosity values is preferably relate to viscosity when determined at 20° C. and 1 atm. In this context, the term “about” is intended to mean±20%, 15%, 10%, 5%, 4%, 3%, 2% or 1% of the stated value.

The viscosity of a vegetable oil increases with increasing saturation, and/or with decreasing temperature Accordingly, saturated vegetable oils with high viscosity, or vegetable oils with high viscosity in which the fatty acids components contain less than 5, 4, 3, 2 or 1 unsaturated bonds per fatty acid chain, may be particularly suitable for the practice of the present invention.

However, although the present invention is particularly suitable for processing high viscosity FAE, it can also be usefully applied to FAEs with lower viscosity, such as less than 200 cP. For example, as described in one embodiment below, the present invention can be applied to the processing of low viscosity oils, such as corn oil, or rapeseed oil, which have a viscosity of about 20-30 cP.

Common vegetable oils or fats suitable for use in the present invention, in addition to or instead of castor oil, can include olive oil, coconut oil, soybean oil, corn oil, palm oil, canola or rapeseed oil, sunflower seed oil, peanut oil, cottonseed oil, palm kernel oil, grape seed oil, vernonia oil, hazelnut and other nut oils, flaxseed/linseed oil, rice bran oil, safflower oil, sesame oil.

Another FAE of particular interest for the practice of the present invention is canola or rapeseed oil for the production of biodiesel. For biodiesel production, the process improvements over existing plant designs also includes the ability to react either methanol or ethanol, or a blend of both to make custom diesel fuels over a range of BTU content and cloud points.

As used herein, the term “fatty acid” is used to refer to “a free fatty acid” and optionally may, or may not, include a “fatty acid derivative.” The term “free fatty acid” means a carboxylic acid having the formula RCOOH. R represents n aliphatic group, preferably an alkyl group. R can comprise an even number between about 2 and about 28 carbon atoms, such as 2, 4, 6, 8, 10, 12, 14, 16, 18, 20, 22, 24, 26 or 28 carbon atoms. Fatty acids can be saturated, monounsaturated, or polyunsaturated (such as up to 5, 4, 3 or 2 unsaturated bonds).

A fatty acid of particular interest for the practice of the present invention is ricinoleic acid, which is the major fatty acid component of castor oil, and is formally called 12-hydroxy-9-cis-octadecenoic acid. Ricinoleic acid has the following structure as shown by Formula I:

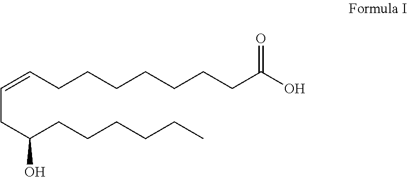 [SMILES: CCCCCC[C@@H](O)C/C=C\CCCCCCCC(=O)O]

Typically, castor oil contains approximately 87% ricinoleic acid by weight, although for the purpose of the practice of the present invention castor oils with difference percentages of ricinoleic acid may be used instead, such as at least 50%, 60%, 70%, 80%, 81%, 82%, 83%, 84%, 85%, 86%, 87%, 88%, 89%, 90%, 81%, 92%, 93%, 94%, 95%, 96%, 97%, 98%, 99% or 100% ricinoleic acid.

Fatty acids are used commercially as surfactants. Surfactants can be found in detergents and soaps. Fatty acids can also be used as additives in fuels, lubricating oils, paints, lacquers, candles, food oils, shortenings, cosmetics, and emulsifiers. Fatty acids can also be used as a feedstock to produce methyl esters, amides, amines, acid chlorides, anhydrides, ketene dimers, and peroxy acids and esters.

The process of the present invention combines and reacts FAE and/or fatty acid in the presence of water and an inorganic base, to produce one or more cleavage products. In the case of one preferred embodiment, in which castor oil and/or ricinoleic acid is reacted with sodium hydroxide, the cleavage products may include sebacic acid and/or 2-octanol. The cleavage products obtained from the reaction of inorganic base with other FAEs and/or fatty acids may be different.

As used herein, biodiesel, an alternative fuel, is comprised of esters (e.g., fatty acid methyl ester, fatty acid ethyl esters, etc.). These esters also have many other commercial uses. Some low molecular weight esters are volatile with a pleasant odour which makes them useful as fragrances or flavouring agents. In addition, esters are used as solvents for lacquers, paints, and varnishes. Furthermore, some naturally occurring substances, such as waxes, fats, and oils are comprised of esters. Esters are also used as softening agents in resins and plastics, plasticizers, flame retardants, and additives in gasoline and oil. In addition, esters can be used in the manufacture of polymers, films, textiles, dyes, and pharmaceuticals.

The castor oil derivatives and/or products which can be derived using processes according to the present invention include: 12 Hydroxy stearic acid, 2 Heptanol, 2 Octanol, Allyl undecylenate, Blown castor oil, Calcium undecylenate, Dehydrated castor oil, Dihydroxystearic acid, Ethoxylated castor oil, Ethyl undecylenate, Heptaaldehyde, Heptanoic acid, Hydrogenated castor, Industrial grade castor oil, Linoleic acid, Methyl ricinoleate, Methyl undecylenate, Nylon 11, Nylon 6, Nylon 6,10, Nylon 6,12, Nylon 6,6, Nylon 9, Oleic acid, Palmitic acid, Pharmacopeia grade castor oil, Ricinoleic acid, Sebacic acid, Sodium undecylenate, Stearic acid, Sulfonated castor oil, Undecanoic acid, Undecylenic acid, Undecylenic alcohol, Undecylenic aldehyde, and Zinc undecylenate.

The present invention will now be further described. In the following passages different aspects of the invention are further defined in more detail. Each aspect so defined may be combined with any other aspect or number of aspects unless clearly indicated to the contrary. Each aspect is described with respect to various different embodiment, and each embodiment so defined may be combined with any other embodiment or number of embodiments for the same aspect unless clearly indicated to the contrary. In particular, any feature indicated as being preferred or advantageous may be combined with any other feature or features indicated as being preferred or advantageous.

The approach of the present invention is a combination of mechanical and chemical improvements to lower the reaction temperatures and also to increase the uniformity of fluid temperatures. The approach provides numerous benefits over prior art processes.

For example, aspects of the present invention permit the process to be efficiently conducted without the presence of a catalyst, such as a toxic heavy metal catalyst, thereby reducing the environmental impact of the process.

Aspects of the present invention provide an alternative approach to managing high viscosity reactants, especially high viscosity oils such as castor oil, without the use of diluents or other non-reactive processing aids, such as mineral oil, PEG, waxes and paraffin, and thereby also avoid the need for downstream processing steps to separate diluents and/or other processing aids from the desired products, which simplifies the process and reduces production costs.

Aspects of the present invention provide an approach to mixing reactants especially high viscosity oils such as castor oil, which do not rely on the use of high powered mixers and agitators, thereby avoiding the expense and maintenance difficulties commonly associated with such mixers.

Importantly, aspects of the present invention also permit a very even temperature distribution within a reaction vessel, thereby avoiding the problems of temperature differentials and localised hotspots which can lead to fluctuations in process parameters and the charring of oil or other FAEs.

Initially, although the present invention will be described predominantly in the context of one of the particularly preferred embodiments, which is the reaction of castor oil with an inorganic base to produce sebacic acid, it will be appreciated that the present invention is not so limited, and other reactants may be substituted in place of the inorganic base to result in the conversion of castor oil into different products, or other starting materials may be substituted in place of castor oil and its reactants in order to produce a different product.

### The First Step: Oil Pre-Heating and Hydrolysis

In a first aspect, the present invention provides for the first step of a method for the treatment of castor oil or other FAE, especially high viscosity FAE, in a reaction vessel, wherein—
the reaction vessel comprises one or more hollow tubes or other conduits, wherein the hollow tubes or other conduits each comprise a first step entry portal, a first step exit portal, and a lumen positioned between the exit and entry portals, and
the treatment of castor oil, or other FAE, occurs in a stream which continuously flows, from the first step entry portal to the first step exit portal, though the, or each, lumen of the one more tubes or other conduits.

Although not a highly viscous FAE, the foregoing treatment may also be useful for pre-treating canola or rapeseed oil, for example, in a preheating step for the production of biodiesel as described further below.

A continuous flow from the first step entry portal to the first step exit portal, through the lumen of a tube or other conduit in this first step, preferably requires the continual unidirectional flow through the lumen, along at least the part of the length of the tube or conduit which is disposed between the first step entry portal to the first step exit portal. This is distinct from a ‘one pot’ reaction vessel which contains reactants that are stirred, but remain, within a single region of the reaction vessel.

The continuous flow through the lumen of the one or more hollow tubes or other conduits in this first step ensures that the initially introduced castor oil, canola or rapeseed oil, or other FAE, is spatially separated, within the reaction vessel, from the downstream treated FAE and/or reaction product(s) generated therefrom. In other words, unlike traditional ‘one pot’ reaction vessels used in the art, such as in the treatment of castor oil, the continuous flow through the tube or other conduit of the reaction vessel result in the separation of initially introduced untreated castor oil, or other FAE, from the downstream treated FAE and/or reaction product(s) generated therefrom.

This permits the addition of further treatment and/or reaction components, into the continuously flowing stream, at spatially separated positions along the length of the one or more hollow tubes or other conduits. In doing so, the contents of the continuous flow can be put through a controlled series of treatment and/or reaction steps, and the steps can be spatially separated.

The reaction vessel used in the first step may comprise multiple tubes, or other conduits, in which the same continuous flow treatment occurs. In the case of multiple tubes, or other conduits, the arrangement may be for parallel flow, or counter flow. In this context, “multiple” includes the meaning more than one, such as up to, or at least, 2, 3, 4, 5, 6, 7, 8, 9, 10, 15, 20, 25, 30, 40, 50, 60, 70, 80, 90, 100, about 150, about 200, about 250, about 300, about 350 or about 400 tubes, or other conduits, may be simultaneously used for the same continuous flow treatment in the reaction vessel. The skilled person will appreciate that the exact number to be used is simply a matter of desired scale, and fewer or more tubes, or other conduits, can be selected as desired. Thus, for example, multiple tubes or conduits can include the meaning of 2-400 tubes or conduits, or even more. The length of each tube may be in the range of 20 to 40 feet or multiples thereof. Typically, the length of each tube used has a practical constraints based, for example, on the shipping length of flat-bed trucks used to deliver the tubes to heat exchanger fabrication shops. Using either welding or couplings the length can be further extended as desired.

The internal measurement of the lumen of the hollow tubes or other conduits used in the first step is typically about 100 mm, about 90 mm, about 80 mm, about 70 mm, about 60 mm, preferably no more than about 55 mm, about 50 mm, about 45 mm, about 40 mm, about 35 mm, about 30 mm, or about 25 mm in at least one dimension. In this context, the term “about” is intended to mean±5, 4, 3, 2, or 1 mm. Where the lumen of the hollow tubes or other conduits is not perfectly circular, the above stated dimension preferably refers to the shortest internal measurement of the lumen.

In the case that viscous FAE is to be treated (such as FAE having a viscosity of greater than 200 cP, 300 cP, 400 cP, 500 cP, about 600 cP or more) then it is particularly preferred that the hollow tubes or other conduits used in the first step are typically capable of withstanding high internal pressures, for example at least about 200 psi, about 300 psi, about 400 psi, about 500 psi, about 600 psi, about 700 psi, about 800 psi, about 900 psi, about 1000 psi, or more. In this context, the term “about” includes them meaning of ±50, 40, 30, 20, 10, 5, or less than 5 psi.

In a particularly preferred embodiment, the lumen of the, or each, tube or other conduit, within the reaction vessel used in the first step, through which the continuous flow occurs, will contain one or more static mixers. Multiple static mixers, presented in series, may be particularly suitable. For example, one static mixer per tube length, prior to a U-turn or bend, may be appropriate for adequate mixing.

Static mixers, for example made of stainless steel, are used to achieve mixing while operating at the often high reaction temperatures required during fluid pumping. The use of static mixers for mixing in-line is an excellent way to achieve low cost mixing predicated on a design targeted for the right viscosity.

The static mixers do not require electrical power and can operate safely at high temperatures and pressures. This permits vigorous mixing without the use of moving parts within the reaction vessel itself. In particular the static mixers do not require a mixing motor, shaft or blades.

Static mixers such as those sold by Komax® normally find applications in mixing a wide range of food products. These include “solid” materials such as margarine and tomato pastes, viscous liquids like syrups, and light fluids similar to soft drink products. To the knowledge of the inventor, static mixers have not, heretofore, been used in the treatment of FAEs.

Komax® static mixers, and equivalents thereof, do not involve any moving parts and elements can be easily removed for external cleaning or used in clean in place (CIP) applications. These types of mixers are powered only by the proportioning pumps in the tube or pipe line system. Substantial power savings and process simplification advantages are achieved over conventional batch mixing techniques.

The combination of the selected lumen dimensions and the presentation of one or more static mixers therefore achieves the efficient mixing of the components of the continuous flow as, during the first step, it passes through the lumen of the reaction tube or other conduit. One particular benefit of this includes the avoidance of powered mixers or agitators within the lumen of the first step reaction vessel.

A further important benefit is the uniform temperature distribution of the components of the continuous flow within the lumen. This is an important benefit over traditional ‘one pot’ reaction vessels used for treatment of castor oil, canola or rapeseed oil, and other FAEs, which typically show a lack of temperature uniformity throughout the reaction components due to difficulty in rapid and complete mixing and/or require the presence of diluents or the like to assist in mixing.

Accordingly, it may be preferred that the temperature of the continuously flowing stream during the first step varies across the cross-section of the lumen of the tube or other conduit by no more than 10° C., 8° C., 6° C., 4° C., 2° C., 1.5° C., 1° C., 0.9° C., 0.8° C., 0.7° C., 0.6° C., 0.5° C., 0.4° C., 0.3° C., 0.2° C., 0.1° C., or less than 0.1° C., at any given point along the length of the tube or other conduit.

In one embodiment, the reaction vessel used in the first step may be, or possess the general configuration of, a heat exchanger, such as a tube and shell, or a plate, heat exchanger. Accordingly, in such an embodiment, the reaction vessel will comprise multiple hollow tubes or other conduits, positioned adjacent to each other to permit the transfer of heat energy, and the continuous flow in the tubes may be arranged to provide parallel flow or counter flow between adjacent tubes or other conduits. In that embodiment, the treatment of castor oil, canola or rapeseed oil, or other FAE, occurs in a stream which continuously flows through the lumens of the tubes or other conduits of the heat exchanger.

According to one embodiment of the first aspect of the present invention, in a first step, the castor oil, canola or rapeseed oil, or other FAE, is introduced, at the first step entry portal, into the lumen of a tube or conduit of the reaction vessel, generally at atmospheric or room temperature, and at a pressure greater than atmospheric pressure. Typically around 10 to 20 bar is suitable for castor oil or other viscous FAE having an equivalent viscosity of about 600 cP. The pressure can be varied dependent on the viscosity of the FAE that is being used. In the case of canola or rapeseed oil which has a much lower viscosity, a pressure of around 3 to 5 bar may be suitable

Previously, the pumping of viscous FAEs has not been practical in FAE treatment methods, but the inventor has realised that pumps that have previously been used for pumping crude oil in oil refineries by the petroleum industry can be suitable for this purpose. These pumps have, to the inventor's knowledge, heretofore not been used outside of the crude petroleum oil industry, and are characterised by operation at both high pressures and high temperatures. Preferably, the pumps operate at a pressure from 1 to 30 bar, or even higher, and temperatures from 100 to 400° C. The pumps have been developed for oil refineries. For example, Hydrodyne offer specially designed vertical high pressure pumps for very high working pressures up to 450 bars.

Accordingly to one embodiment, after having been pumped into the lumen of a tube or conduit of the first step reaction vessel under pressure, and during its movement in a continuous flow through the lumen, the castor oil, canola or rapeseed oil, or other FAE, is exposed to high pressure steam which is used to heat the castor oil, canola or rapeseed oil (or other FAE).

The steam may be introduced into the lumen of the tube or conduit of the reaction vessel, via one or more steam entry portals.

The one or more steam entry portals are positioned at one or more locations upstream or, more preferably downstream, of the first point of entry of the castor oil, canola or rapeseed oil, or other FAE, via the first step entry portal, into the lumen of the tube or conduit of the reaction vessel. As a consequence, the steam mixes with the continuous flow of castor oil, canola or rapeseed oil, or other FAE, within the lumen. The mixing may preferably be assisted by the suitable positioning of one or more static mixers within the lumen of the tube or conduit of the reaction vessel at one or more positions downstream of the, or each, of the steam entry portals.

The rate of flow of the castor oil, canola or rapeseed oil, or other FAE, and steam mixture over the static mixers needs to be turbulent flow.

A target temperature, resulting from the mixing of the heated FAE and steam will be selected based on the identity and requirements of the FAE. For example, when the highly viscous castor oil is used, the target temperature may be greater than about 200° C., such as more than about 250° C., preferably within the range of about 300° C. to about 320° C. Temperatures higher than about 320° C. are generally not desirable with castor oil due to the charring of the castor oil and the consequent creation of tar which can block the apparatus. In contrast, when the much less viscous canola or rapeseed oil is used, a target temperature of about 50-60° C. may be suitable. Other target temperatures may be suitable for other FAEs, and such temperatures may be determined empirically using routine tests.

The effect of the heated and pressured steam treatment of the castor oil, canola or rapeseed oil, or other FAE, according to this first step can include a reduction in viscosity.

A further effect of the heated and pressured steam treatment of the castor oil, canola or rapeseed oil, or other FAE, according to this first step can be he hydrolysis of castor oil, canola or rapeseed oil, or other FAE, to release fatty acids (especially ricinoleic acid in the case of the hydrolysis of castor oil) and an alcohol component (typically glycerol).

The products of the first step may, therefore, comprise, consist essentially of, or consist of a mixture of, castor oil (or other FAE) as initially introduced to the lumen, and water. The products of the first step may further comprise the reaction products of the hydrolysis of the castor oil (or other FAE), i.e., free fatty acids and an alcohol component. Suitable ratios of these components may, for example, be from 1.2 to 1.5 to 1.0 by weight.

Typically, the target temperature and pressure used to treat a low viscosity FAE, like canola or rapeseed oil, during this first step, in a process for the production of biodiesel, will not result in any, or any substantial levels, of hydrolysis or viscosity reduction. In that case, the first step may be simply performed as a pre-heating step.

The products of the first step exit via the first step exit portal at high temperature, ideally a temperature that is the same as the target temperature defined above; and at high pressure. The temperature of the product of the first step will typically be higher than the temperature of the FAE that enters through the first step entry portal. The viscosity of the product of the first step will typically be lower than the viscosity of the FAE that enters through the first step entry portal. The water content of the product of the first step will typically be higher than the water content of the FAE that enters through the first step entry portal. There may be an increase in the ratio of free fatty acids to FAE in the product of the first step compared to the ratio in the FAE that enters through the first step entry portal.

The product of the first step may then be used in a second step as described further below.

Alternatively, although less preferably, the first step may be entirely bypassed, by providing the same products, at the same temperature, viscosity and/or water content via an alternative process or from an alternative source.

In either case, these products can be presented for a second step, as described below, at high temperature, ideally a temperature that is the same as the target temperature defined above (typically about 300° C. to 320° C. in the case of castor oil and/or ricinoleic acid; typically about 50-60° C. in the case of canola or rapeseed oil); and at high pressure

The Second Step: Reaction with an Inorganic Base

The second step involves the reaction of a FAE and/or fatty acid, with an inorganic base, in the presence of water. In one embodiment of particular interest, the FAE is castor oil and/or the fatty acid is ricinoleic acid. In another embodiment of particular interest, the FAE is canola or rapeseed oil.

Whereas the reaction of castor oil in a strongly basic solution under heat is well known in the art for the production of sebacic acid and 2-octanol, the present inventor has developed a new and improved process for reacting these components.

As indicated above, the FAE (such as castor oil, or canola or rapeseed oil) and/or fatty acid (such as ricinoleic acid) may most conveniently and efficiently be provided by the above-described first step, or it may be provided via an alternative process or from an alternative source.

With the production of sebacic acid and 2-octanol, from castor oil (or from another source of ricinoleic acid), the process of the second step combines castor oil and/or ricinoleic acid with an inorganic base in the presence of water. Ideally, although not necessarily, the castor oil and/or ricinoleic acid is provided by a first step as described above.

Thus, the process of the second step combines FAE (such as castor oil, canola or rapeseed oil) and/or the fatty acid (such as ricinoleic acid) with an inorganic base in the presence of water, in a reaction vessel.

An important development in this second step of the process, compared to the prior art, is the methodology used to combine the FAE (such as castor oil) and/or the fatty acid (such as ricinoleic acid) with an inorganic base in the presence of water.

The inventor has realised that these components can be efficiently reacted, in the second step, without the use of transition metal catalyst traditionally used in sebacic acid production, and without the need for the addition of non-reactive diluents or other non-reactive processing aids to reduce the viscosity of the reaction mixture typically required by viscous FAEs such as castor oil, by a second aspect of the present invention, which provides a process for the reaction of a FAE and/or a fatty acid with an inorganic base comprising the following steps—
(i) providing a pressurised and heated stream of the FAE and/or fatty acid;
(ii) providing an inorganic base; and
(iii) combining the stream obtained in step (i) with the inorganic base obtained in step (ii), in the presence of water, in a second step reaction vessel, by injection;
(iv) optionally maintaining the stream obtained in step (i) at a selected temperature and pressure; and
(v) thereby obtaining a reaction product.

As discussed above, the pressurised stream of the FAE and/or fatty acid at increased temperature may most conveniently and efficiently be provided by the above-described first step according to the first aspect of the present invention, or it may be provided via an alternative process or from an alternative source. Accordingly, it is envisaged that the second step process of the second aspect of the invention may be performed in combination with, or independently of, the first step process of the first aspect of the invention.

Water may be present as part of the pressurised stream of the FAE and/or fatty acid, and/or introduced in conjunction with the inorganic base and/or added separately. Following the injection defined by step (iii) above, the water will generally be present in the form of steam, although in practice it may comprise a mixture of water in liquid and gaseous phases, and can include super-heated liquid water, mist, water vapour and/or steam.

The inorganic base may be provided in the form of a powder, or as a solution (preferably wherein the inorganic base is present in solution at a concentration (w/w) of about 40%, about 50%, about 60%, about 70% or more, wherein the term “about” refers to ±5, 4, 3, 2, or 1%). In either case, it may be combined with pressurised and heated stream of the FAE and/or fatty acid by injection into the pressurised and heated stream of the FAE and/or fatty acid provided by step (i). Thus, for example, the reaction vessel contains the pressurised and heated stream of the FAE and/or fatty acid, and the inorganic base is injected into that stream at one or more injection points. In an alternative embodiment, the inorganic base may be powdered, and blended with the FAE and/or fatty acid prior to its entry into the second step reaction vessel, in which case the resultant mixture may be injected into the reaction vessel to generate a stream, and/or the reaction vessel contains a pressurised and heated stream comprising a mixture of the FAE and/or fatty acid with the inorganic base, and pressurised and heated water (typically in the form of vapour, such as steam) is injected into the stream.

Thus, the process of the second step involves the injection of at least one, two or all three of the inorganic base, pressurised and heated water, and/or the pressurised and heated stream of the FAE and/or fatty acid into the reaction vessel.

Where the inorganic base is introduced into the pressurised and heated stream of the FAE and/or fatty acid as a powder, then the pressurised and heated stream of the FAE and/or fatty acid will also contain water and/or have further water added thereto. This permits the powdered inorganic base to be combined with the water during the reaction process.

In another embodiment, the inorganic base may be combined with water prior to the injection defined by step (ii) above. For example, a hot inorganic base preparation may be generated by combination of the inorganic base with steam, or by combination of water followed by heating.

The temperature of the inorganic base preparation, as it is injected during step (iii) into the reaction vessel and combined with the pressurised and heated stream obtained from step (i), will depend on the desired temperature of reaction between the FAE and/or fatty acid, the water, and the inorganic base. Where the FAE is castor oil and/or the fatty acid is ricinoleic acid, then it may be appropriate to heat the inorganic base to between about 300° C. to about 340° C. prior to injection. Where the FAE is canola or rapeseed oil, then it may be appropriate to heat the inorganic base to between about 50-60° C. prior to injection. In this context, the term “about” includes the meaning±20° C., 15° C., 10° C., 5° C. or less.

Any suitable inorganic base may be used. The appropriate base can be selected by the skilled person, dependent on the identity of the FAE and/or fatty acid and dependent on the desired product. For example, the inorganic base may be a metal hydroxide, such as sodium hydroxide or potassium hydroxide, or a metal methoxide such as sodium methoxide. Sodium hydroxide may be particularly suitable for use in conjunction with castor oil and/or ricinoleic acid in the production of sebabcic acid and/or 2-octanol. Sodium hydroxide, potassium hydroxide, or sodium methoxide may be particularly useful in conjunction with canola or rapeseed oil, or other FAEs, in the production of biodiesel. Sodium hydroxide tends to be generally less expensive than, for example, potassium hydroxide.

Accordingly, in a preferred embodiment, especially when the fatty acid is ricinoleic acid and the desired end product is sebacic acid, the inorganic base is sodium hydroxide.

In one embodiment, sodium hydroxide is presented as a powder which can be added directly into a stream of castor oil and/or ricinoleic acid which contains some process water and can be blended to be uniform. The blending can occur prior to the entry of the blended mixture into the second step reactor vessel and may even occur prior to a first pre-heating step. The blended mixture can then be injected into the second step reaction vessel. Direct steam injection may be used (either before and/or after entry of the blended mixture into the second step reaction vessel) to heat the oil and achieve the heat of solution reaction, which may also generate additional heat as the sodium hydroxide is further diluted.

In another embodiment, the sodium hydroxide (either in powdered or solution form) is injected directly into the second step reactor vessel to combine with a pressurised and heated stream of castor oil and/or ricinoleic acid contained therein.

In another embodiment, sodium hydroxide, potassium hydroxide, or sodium methoxide may be particularly useful in conjunction with canola or rapeseed oil, or other FAEs, in the production of biodiesel.

In processes for the production of biodiesel from canola or rapeseed oil, or other FAEs, the inorganic base and FAE are also reacted in the presence of a C₁-C₃ mono-alcohol, such as methanol, ethanol or propanol (or, optionally, in the case that sodium methoxide is used, then it may not be necessary to provide a C₁-C₃ mono-alcohol). Ethanol and methanol may be preferred, and methanol may be most preferred. The C₁-C₃ mono-alcohol may be pre-mixed with the inorganic base prior to its addition to the heated and pressurised FAE stream, or may be added to the heated and pressurised FAE stream as a separate injection from the inorganic base. Thus, the C₁-C₃ mono-alcohol and the inorganic base may be added to the heated and pressurised FAE stream simultaneously or sequentially.

As discussed above, the process of the second step involves the injection of at least one, two or all three of the inorganic base, pressurised and heated water, and/or the pressurised and heated stream of the FAE and/or fatty acid into the second step reaction vessel. Optionally, in the case of biodiesel synthesis, the C₁-C₃ mono-alcohol may alternatively or additionally be injected into the second step reaction vessel.

The second step of the process, as defined by the second aspect of the invention, preferably uses at least one sonic nozzle, or a series of sonic nozzles, to inject at least one, two, three or all four of the inorganic base, optional C₁-C₃ mono-alcohol, pressurised and heated water (e.g., steam), and/or the pressurised and heated stream of the FAE and/or fatty acid into the second step reaction vessel. The use of one or more sonic nozzles dramatically reduces reaction times compared to other forms of injection. The sonic nozzles also assist with complete mixing of the reaction components in the reaction vessel.

Sonic nozzles are well known and commonly available, although to the inventor's knowledge there has been no previous disclosure of their use in the mixing of FAE and/or fatty acid with an inorganic base.

Typically, a sonic nozzle comprises a smooth rounded inlet section converging to a minimum throat area and then diverges along a pressure recovery section or exit cone. As gas or particulate matter (e.g., misted liquids, aerosols, particles, powders) accelerates through the nozzle, its velocity increases and its density decreases. The maximum velocity is achieved at the throat, the minimum area where it achieves the desired speed of sound. An example of a suitable sonic nozzle is one sold by the company TrigasFI GmbH.

The sonic nozzle is operated by pressurizing the inlet or evacuating the exit to achieve and inlet/outlet pressure ratio of 1:4 or greater. This ratio maintains the nozzle in a “sonic” state. In this state, only the upstream pressure and the temperature influence the flowrate through the nozzle. The flowrate therefore becomes nearly an inlet function of the linear pressure. Doubling the inlet pressure doubles the flowrate. Therefore the pressure differences within a piping system travel and the speed of sound and generate flow.

The simplest flow system uses an inlet pressure regulator to control air pressure and a thermocouple to measure the temperature. Adjusting the pressure regulator will change and maintain the flow through the nozzle.

It is noted that sonic nozzles are extremely noisy, and that noise reduction apparatus (such as acoustic baffling) may be advantageously employed to reduce the noise to a safe and comfortable level, such as below 70 dB.

The use of sonic nozzles, or other high speed injectors, for introducing at least one, two, three, or all four of the inorganic base, the optional C₁-C₃ mono-alcohol, the pressurised and heated water (e.g., steam), and/or the pressurised and heated stream of the FAE and/or fatty acid into the second step reaction vessel ensures high levels of mixing, and this provides numerous advantages.

Components injected using sonic nozzles create microbubbles, which expand and collapse, and create sonic mixing within the reaction mixture. This reduces the reaction time, and increases the reaction efficiency, and reduces the necessary reaction temperature. In the case of, for example, the reaction of castor oil and/or ricinoleic acid, a reduced reaction temperature can be very important in avoiding temperatures which cause charring of the oil.

Furthermore, the use of one or more sonic nozzles in accordance with the present invention achieves low ratios of inorganic base to FAE and/or fatty acid, thus providing a process which is able to convert castor oil to sebacic acid using lower sodium hydroxide to castor oil ratios compared to the ratios used in existing commercial plants and previously-know processes for producing sebacic acid.

In the castor oil reaction, the molar ratio of sodium hydroxide to castor oil may be from 1:1 to 3:1, such as less than 3:1, less than 2.5:1, less than 2:1, or less than 1.5:1.

In the biodiesel oil reaction, the molar ratio of sodium hydroxide to canola or rapeseed oil may be about 1.5:1, such as from about 2.5:1 to 1:1, or 2:1 to 1:1, most preferably 1.5(±0.4, 0.3, 0.2 or 0.1):1.

Accordingly, by using one or more sonic nozzles to inject the inorganic base (such as sodium hydroxide), the amount of inorganic base required to achieve sufficient reaction rates can be lowered, thereby reducing the cost of the process.

Furthermore, the invention avoids the use of catalysts, in particular heavy metal catalysts which comprise, for example, lead, cadmium, bismuth, titanium and other such heavy metals. Accordingly, a particularly preferred embodiment of the present invention provide a process according to the second aspect of the present invention, particularly one which utilises castor oil and/or ricinoleic acid, and which does not include the addition of a heavy metal catalyst which comprise, for example, lead, cadmium, bismuth, titanium and other such heavy metals.

A further advantage associated with the use of sonic nozzles, or other high speed injectors, is that it avoids the need for high powered mixers and agitators within the reaction vessel. The injection can be used and controlled with no moving parts except the recirculation pump and air actuated steam valves, which are located outside of the reaction vessel and thus less exposed to reaction conditions, and more easily accessed for maintenance.

Accordingly, in one embodiment, the second step reaction vessel contains no moving parts within the lumen in which the reaction occurs and, more particularly, contains no moving mixers.

In one embodiment, instead of one sonic nozzle, a series of sonic nozzles can be used to both increase the temperature and inject steam under pressure setting up a sonic wave for mixing and reaction.

As indicated above, the pressurised and heated stream of FAE (such as castor oil, or canola or rapeseed oil) and/or the fatty acid (such as ricinoleic acid), the pressurised stream of inorganic base, optionally the C₁-C₃ mono-alcohol, and water are reacted together, during the second step, within in a reaction vessel. In one preferred embodiment:
the reaction vessel, as used in the second step of the process, may comprises one or more hollow tubes or other conduits wherein the hollow tubes or other conduits each comprise a second step entry portal, and a second step exit portal, and a lumen positioned between, and
the second step reaction occurs in a stream which continuously flows, from the second step entry portal to the second step exit portal, though the, or each, lumen of the one more tubes or other conduits.

As will be readily apparent, the reaction vessel used in the second step may be the same as, and continuous with, the reaction vessel used in the first step. Thus, for example, the reaction vessels used in the first and second steps of the process may be a continuous tube or other conduit, and the first step exit portal may be the same as the second step entry portal.

Alternatively, the reaction vessel used for the second step may not be the same as the reaction vessel used in the first step (if any first step is conducted at all, which may not be the case if the products of the first step are derived from a different source). If different reaction vessels are used for the first and second steps, then they may be suitably connected to permit the transfer of the products exiting the first step exit portal to the second step entry portal, with or without intervening steps or treatments.

In the same way as discussed above in respect of the first step, and equally applicable to the second step, a continuous flow from the second step entry portal to the second step exit portal, through the lumen of a tube or other conduit in this second step preferably requires the continual unidirectional flow through the lumen, along at least the part of the length of the tube or conduit which is disposed between the second step entry portal to the second step exit portal. This is distinct from a ‘one pot’ reaction vessel which contains reactants that are stirred, but remain, within a single region of the reaction vessel.

The continuous flow through the lumen of the one or more hollow tubes or other conduits in this second step ensures that the FAE and/or fatty acid that is introduced at the second step entry portal is spatially separated, within the reaction vessel, from the downstream reaction product(s). In other words, unlike traditional ‘one pot’ reaction vessels used in the art for treatment of castor oil, the continuous flow through the tube or other conduit of the reaction vessel result in the spatial separation of initially introduced FAE and/or fatty acid from the downstream product(s).

This permits the addition of further reaction components (in particular, inorganic base and/or water, and optionally C₁-C₃ mono-alcohol), during the second step of the process, into the continuously flowing stream, at one or more spatially separated positions along the length of the one or more hollow tubes or other conduits between the second step entry portal and second step exit portal. In doing so, the contents of the continuous flow can be put through a controlled reaction step, or series of reaction steps, during the second step of the process, and the steps can be spatially separated within the lumen.

Accordingly, the second step entry portal permits the entry of the pressurised and heated stream of FAE (such as castor oil, or canola or rapeseed oil) and/or the fatty acid (such as ricinoleic acid) into the lumen of the one or more hollow tubes or other conduits of the second step reaction vessel. As indicated above, the second step entry portal may be one or more sonic nozzles or other high speed injectors. Alternatively, the second step entry portal may be any other form of entry portal which permits the entry of a continuously flowing pressurised and heated stream of FAE and/or fatty acid into the lumen of the one or more hollow tubes or other conduits of the second step reaction vessel. In that case, at least one other component selected from the inorganic base and/or water, and optionally C₁-C₃ mono-alcohol, may be injected into the stream of FAE and/or fatty acid via one or more sonic nozzles or other high speed injectors

The reaction vessel used in the second step may comprise multiple tubes, or other conduits, in which the same continuous flow treatment occurs. In the case of multiple tubes, or other conduits, the arrangement may be for parallel flow, or counter flow. In this context, “multiple” includes the meanings more than one, such as up to, or at least, 2, 3, 4, 5, 6, 7, 8, 9, 10, 15, 20, 25, 30, 40, 50, 60, 70, 80, 90, 100, about 150, about 200, about 250, about 300, about 350 or about 400 tubes, or other conduits, may be simultaneously used for the same continuous flow treatment in parallel in the reaction vessel. The skilled person will appreciate that the exact number to be used is simply a matter of desired scale, and fewer or more tubes, or other conduits, can be selected as desired. Thus, for example, multiple tubes or conduits can include the meaning of 2-400 tubes or conduits, or even more. The length of each tube may be 5 to 50 meters

The internal measurement of the lumen of the hollow tubes or other conduits used in the second step is typically about 100 mm, about 90 mm, about 80 mm, about 70 mm, about 60 mm, preferably no more than about 55 mm, about 50 mm, about 45 mm, about 40 mm, about 35 mm, about 30 mm, or about 25 mm in at least one dimension. In this context, the term “about” is intended to mean±4, 3, 2, or 1 mm. Where the lumen of the hollow tubes or other conduits is not perfectly circular, the above stated dimension preferably refers to the shortest internal measurement of the lumen.

The hollow tubes or other conduits used in the second step, particularly with the treatment of viscous FAE such as castor oil, are typically capable of withstanding high internal pressures, for example at least about 200 psi, about 300 psi, about 400 psi, about 500 psi, about 600 psi, about 700 psi, about 800 psi, about 900 psi, about 1000 psi, or more. In this context, the term “about” includes them meaning of ±50, 40, 30, 20, 10, 5, or less than 5 psi.

The lumen of the, or each, tube or other conduit, within the reaction vessel used in the second step, through which the continuous flow occurs, may contain one or more static mixers. Multiple static mixers, presented in series, may be particularly suitable.

Preferred static mixers may be as described above in respect of the first step.

The combination of the selected lumen dimensions and the presentation of one or more static mixers therefore achieves the further efficient mixing of the components of the continuous flow as, during the second step, it passes through the lumen of the reaction tube or other conduit. One particular benefit of this includes the avoidance of powered mixers or agitators within the lumen of the reaction vessel.

The combination of introducing one or more of the reaction components into the lumen of the hollow tubes or other conduits of the second step reaction vessel via one or more sonic nozzles, along with the continuous flow of the resultant through the lumen of a reaction tube or other conduit, especially wherein the lumen contains one or more in-line static mixers, is an especially preferred embodiment of the present invention, due to the highly efficient nature of the mixing that it achieves within the continuous flow.

A further important benefit is the uniform temperature distribution of the components of the continuous flow within the lumen during the second step. This is an important benefit over traditional ‘one pot’ reaction vessels used for treatment of castor oil and other FAEs, which typically show a lack of temperature uniformity throughout the reaction components due to difficulty in rapid and complete mixing and/or require the presence of non-reactive diluents or the like to assist in mixing.

Accordingly, it may be preferred that the temperature of the continuously flowing stream during the second step varies across the cross-section of the lumen of the tube or other conduit by no more than 10° C., 8° C., 6° C., 4° C., 2° C., 1.5° C., 1° C., 0.9° C., 0.8° C., 0.7° C., 0.6° C., 0.5° C., 0.4° C., 0.3° C., 0.2° C., 0.1° C., or less than 0.1° C., at any given point along the length of the tube or other conduit.

In one embodiment, the reaction vessel used in the second step may be, or possess the general configuration of, a heat exchanger, such as a tube and shell, or a plate, heat exchanger. Accordingly, in such an embodiment, the reaction vessel will comprise multiple hollow tubes or other conduits, positioned adjacent to each other to permit the transfer of heat energy, and the continuous flow in the tubes may be arranged to provide parallel flow or counter flow between adjacent tubes or other conduits. In that embodiment, the reaction of the FAE and/or fatty acid with inorganic base in the presence of water occurs in a stream which continuously flows through the lumens of the tubes or other conduits of the heat exchanger. Accordingly the first and/or second steps, as defined by the first and/or second aspects of the invention, respectively, may use a shell or tube heat exchanger as the actual reaction vessel in contrast to the large expensive high pressure vessels. Super-heated steam may, for example, be used in the shell to transfer heat to the reaction components as they pass though the lumens of the tubes or other conduits within the heat exchanger.

The pumps which are used to pump the FAE and the reaction stream in the first and/or second steps, as defined by the first and/or second aspects of the invention, operate at both high pressures and high temperatures. Preferably, the pumps operate at a pressure from and temperatures from Suitable pumps have been developed for oil refineries as used in the petroleum industry. For example, Hydrodyne offer specially designed vertical high pressure pumps for very high working pressures up to 450 bars.

Thus, the second step process results in the reaction of FAE and/or fatty acid with an inorganic base in the presence of water, to generate a cleavage product. Typically, the conditions selected in the second reaction vessel, in particular the temperature, pressure, reaction time, and extent of mixing, will result in the hydrolysis of FAE to generate fatty acid and/or the cleavage of the FAE and/or fatty acid by the inorganic base to generate one of more cleavage products. For example, in the case of the reaction of ricinoleic acid with sodium hydroxide (or other suitable bases), the cleavage products preferably comprise, consist essentially of, or consist of sebacic acid and 2-octanol. In the case of the reaction of canola or rapeseed oil with the C₁-C₃ mono-alcohol (such as methanol) and the inorganic base (such as a sodium hydroxide or potassium hydroxide, the cleavage products preferably comprise, consist essentially of, or consist of fatty acid methyl ester (i.e., biodiesel) and glycerol.

Products recovered from the second step reaction vessel will therefore comprise such cleavage products, optionally mixed with one or more of water, inorganic base, unreacted fatty acid, released alcohol (such as glycerol) and unreacted FAE.

Where FAE is provided as a reactant in the second step, then the second step preferably consumes at least 10%, 20%, 30%, 40%, 50%, 60%, 70%, 80%, 90%, 95%, 96%, 97%, 98%, 99% or more, by weight, of that FAE by chemical conversion. Where fatty acid is provided as a reactant in the second step, then the second step preferably consumes at least 10%, 20%, 30%, 40%, 50%, 60%, 70%, 80%, 90%, 95%, 96%, 97%, 98%, 99% or more, by weight, of that FAE by chemical conversion.

It may be preferred that the products recovered from the second stage reaction vessel comprise cleavage products in a weight ratio with FAE of at least 10:90, at least 20:80, at least 30:70, at least 40:60, at least 50:50, at least 60:40, most preferably at least 70:30, at least 80;20, at least 90:10, at least 95:5, at least 96:4, at least 97:3, at least 98:2, at least 99:1, or greater than 99:less than 1.

It may additionally, or alternatively, be preferred that the products recovered from the second stage reaction vessel comprise cleavage products in a weight ratio with fatty acid of at least 10:90, at least 20:80, at least 30:70, at least 40:60, at least 50:50, at least 60:40, most preferably at least 70:30, at least 80;20, at least 90:10, at least 95:5, at least 96:4, at least 97:3, at least 98:2, at least 99:1, or greater than 99:less than 1.

In the embodiment wherein the second step reaction vessel comprises one or more hollow tubes or other conduits wherein the hollow tubes or other conduits each comprise a second step entry portal, and a second step exit portal, and a lumen positioned between, and the second step reaction occurs in a stream which continuously flows, from the second step entry portal to the second step exit portal, though the, or each, lumen of the one more tubes or other conduits, then—
the path length of the continuous flow, from the second step entry portal to the second step exit portal, may be selected to be of a length suitable to achieve the desired composition of the product as it passes through the second step exit portal; or
in a convenient alternative, a recirculation loop may be provided, to permit the recirculation of reaction components in the continuous flow within the second step reaction vessel, before exit from the second step reaction vessel through second step exit portal. Recirculation may occur one or more times, to the extent necessary to permit the reaction to be driven to the desired completion point. Accordingly, the recirculation loop provides a path for components in the continuous flow, at a position that is downstream of the second step entry portal, to be diverted (at a diversion point) before reaching the second step exit portal, and re-entered (at a re-entry point) into the continuous flow at a point upstream of the diversion point. For example, with reference to the positions of the second step entry portal and the second step exit portal, which define the start and finish, respectively, of the flow path, such that the position of the second step entry portal may be referred to as being 0% along the flow path, and to the second step exit portal may be referred to as being 100% along the flow path, then the diversion point will typically be at a position that is greater than 50%, 60%, 70%, 80%, 90%, or 95% along the flow path and the re-entry point will typically be at a position that is less than 50%, 40%, 30%, 20%, 10%, or 5% along the flow path, although other positions may be used provided that the diversion point is further downstream than the re-entry point.

Accordingly, in one embodiment, the second step reaction is performed as a continuous process. For example, the reaction vessel apparatus can incorporate a continuous feed entry and product exit configuration by adjusting flow rate, length of heated tubes, or other conduits, with the internal static mixers and steam injectors.

In another embodiment, the second step reaction is performed as a batch process. For example, the reaction vessel apparatus may include a recirculation loop, in addition to the a feed entry and product exit configuration by adjusting flow rate, length of heated tubes, or other conduits, with the internal static mixers and steam injectors.

As indicated above, the product that is recovered from the second step reactor vessel will comprise one or more desired cleavage products (such as sebacic acid and octanol-2, in the case that castor oil and/or ricinoleic acid is reacted with sodium hydroxide; or biodiesel and glycerol in the case that canola or rapeseed oil is reacted with a methanol and sodium hydroxide), but will also typically include other components, such as unreacted FAE and/or fatty acid, water, and remaining inorganic base.

In one very important embodiment, the product that is recovered from the second step reactor vessel contains no, or substantially no, transition metal catalysts, such as oxides and/or chlorides of one or more (including all) of lead, bismuth, cadmium and titanium, or any other form of catalyst, especially toxic or environmentally damaging catalysts.

In another very important embodiment, the product that is recovered from the second step reactor vessel contains no, or substantially no (for example, no more than 10%, 5%, 4%, 3%, 2%, 1% by weight), viscosity-lowering non-reactive diluents or non-reactive processing aids, especially any one or more of mineral oil, polyethylene glycol (PEG), waxes and paraffin.

It will be appreciated that the second step process according to the second aspect of the present invention is suitable for use both without the inclusion of catalyst and without the inclusion of viscosity-lowering non-reactive diluents or non-reactive processing aids, and so a particularly preferred embodiment results in a product which contains no, or substantially no, catalyst as defined above and no, or substantially no, viscosity-lowering non-reactive diluents or non-reactive processing aids as defined above.

### The Third Step: Product Separation

As indicated above, the product that is recovered from the second step reactor vessel, via the second step exit portal, contains a mixture of components, including the one or more desired cleavage products, but also typically including one or more of unreacted inorganic base, water, unreacted FAE, unreacted fatty acid, and/or alcohol (such as glycerol).

To obtain the one or more desired cleavage products (such as the sebacic acid and 2-octanol) from the product that is recovered from the second step reactor vessel, one or more, further separation steps is/are generally performed. However, although preferred, this is not a mandatory feature of the second aspect of the present invention.

Any suitable separation step or combination of steps may be used, and will be selected based on the properties of the one or more desired cleavage products for recovery, also taking into consideration the characteristics of the other components, such as the unreacted FAE and/or unreacted fatty acid, alcohol, water, and remaining inorganic base. The skilled person can readily use any of the numerous separation techniques known in the art.

One suitable technique may be distillation. Distillation is a purification technique which would be well known to those skilled in the art. It may, for example, be suitable to use a distillation column which is operated at atmospheric pressure, or under a partial vacuum. In one embodiment, only low cost distillation is required because no additional organic solvents, viscosity-lowering non-reactive diluents or non-reactive processing aids (especially any one or more of mineral oil, polyethylene glycol (PEG), waxes and paraffin) are present in the recovered product. In the case of reaction products derived from reacting FAE with C₁-C₃ mono-alcohol and inorganic base, then unreacted C₁-C₃ mono-alcohol may also be present in the recovered product, and this (especially methanol) may be usefully separated by distillation due to its high vapour pressure.

It may be preferred to use distillation as an initial separation step for the product obtained directly from the second step reactor vessel, via the second step exit portal. Alternatively, it may be used downstream of one or more other separation steps.

The product that is recovered from the second step reactor vessel, via the second step exit portal, or after the distillation step, may contain various side-products, and these can be further separated from the desired cleavage products (such as sebacic acid and/or 2-octanol).

For example, a settling tank can be used, to allow fluid products recovered from the second step reactor vessel, via the second step exit portal, or after the distillation step, to be separated by gravity into a lower aqueous fraction and an upper fraction which is non-miscible with the aqueous fraction. Typically the upper fraction contains FAE, fatty acid, and/or other components which are miscible with FAE and/or fatty acid and/or are poorly soluble in the aqueous fraction. Usually, the desired cleavage products (e.g., sebacic acid and 2-octanol) will be present predominantly or exclusively in the upper fraction.

Thus, in one embodiment, a settling tank may be used to separate the water and oil components.

The inorganic base in the recovered product will be primarily, although not necessarily entirely, present in the aqueous fraction. Any remaining inorganic base in the non-aqueous fraction may be recovered using a washing step, for example using water.

In the case of biodiesel production, the use of a settling tank may be particularly useful to separate biodiesel from glycerol.

An additional advantage of the invention, which derived at least in part from the absence of lead, cadmium, bismuth and other toxic transitional metals in the reaction product, is that the inorganic base (such as sodium hydroxide, potassium hydroxide, sodium methoxide, etc.) can be recovered from the product that is recovered from the second step reactor vessel, via the second step exit portal, or a downstream fraction thereof, such as an aqueous fraction. Suitable techniques for the capture of sodium hydroxide are known in the art and include, for example, the use of ion exchange resins. For example those manufactured by Dow

Alternatively, unreacted inorganic base recovered in the product may be neutralised using acid, typically a dilute acid.

Prior to the present invention, the effective recovery or neutralisation of inorganic base from such treatments (for example, the repeated recovery of sodium hydroxide from conventional methods of converting castor oil to sebacic acid) were not possible due to the accumulation of the lead, cadmium, bismuth and other toxic transitional metal catalysts by the inorganic base. Whereas, with prior art methods, such as those for the production of sebacic acid, the sodium hydroxide could perhaps be recycled a few times, but eventually the catalyst mixture would have to be dumped by into a waste stream, and a fresh catalyst mixture introduced in order to maintain productivity. This is both costly (due to the loss of inorganic base) and environmentally hazardous (due to the release of a toxic catalyst mixture). In particular, the environmental hazards associated with dumping used catalyst mixture in line with prior art processes have prevented new sebacic acid manufacturing plants from being constructed, due to regulatory constraints imposed by national laws, such as in the US. By addressing this problem, the present invention provides a process which does not employ toxic transition metal catalysts and so its waste streams will not cause problematic environmental pollution, thereby opening the opportunity to build new manufacturing plants which comply with regulatory requirements. This is a major improvement on prior art processes which utilised toxic catalysts.

Additionally, any cleaned up and purified water that is recovered during the separation step may be recycled for use in any one or more of the earlier steps of the process.

Typically, a non-aqueous fraction will be recovered, comprising a mixture of the one or more desired cleavage products (e.g., sebacic acid and/or 2-octanol) and some remaining unreacted FAE and/or fatty acids.

Due to the heat treatment during the first and/or second steps, the mixture may comprise some darkened FAE, which can be removed by suitable techniques, for example, nanofiltration or activated charcoal. Previous methods which employed metal catalysts, in particular metal oxide catalysts, were generally not compatible with the use of activated charcoal filters.

In one embodiment of the third step, any unreacted FAE and/or fatty acids obtained at the separation stage may preferably be reintroduced into the process for further reaction, either at the start or mid-way through the first step, or at the start or mid-way through the second step.

Consequently, through one or more separation and purification steps, the one or more desired cleavage products (e.g., sebacic acid and/or 2-octanol; or biodiesel and glycerol) may be isolated and purified. The extent of purification will depend on the quality requirements for the use of the, or each, cleavage product. It may, for example, be preferred that each cleavage product is separated into a form that is at least 50%, 60%, 70%, 80%, 90%, 95%, 96%, 97%, 98%, 99%, 99.1%, 99.2%, 99.3%, 99.4%, 99.5%, 99.6%, 99.7%, 99.8%, 99.9%, 99.99%, 99.999%, or 100% pure, by weight.

Accordingly, for example, the present invention can provide highly pure forms of sebacic acid and/or 2-octanol, as produced by the process of the second aspect of the present invention and subsequently purified according to the third step of separation as discussed above.

In another example, the present invention can provide a highly pure form of biodiesel, as produced by the process of the second aspect of the present invention and subsequently purified according to the third step of separation as discussed above.

### The Fourth Step: Processing the Recovered Cleavage Products

The products obtained from the third step of separation can be further formulated or reacted with other components in order to produce one or more further downstream products.

The castor oil derivatives and/or products which can be derived using processes according to the present invention include: 12 Hydroxy stearic acid, 2 Heptanol, 2 Octanol, Allyl undecylenate, Blown castor oil, Calcium undecylenate, Dehydrated castor oil, Dihydroxystearic acid, Ethoxylated castor oil, Ethyl undecylenate, Heptaaldehyde, Heptanoic acid, Hydrogenated castor, Industrial grade castor oil, Linoleic acid, Methyl ricinoleate, Methyl undecylenate, Nylon 11, Nylon 6, Nylon 6,10, Nylon 6,12, Nylon 6,6, Nylon 9, Oleic acid, Palmitic acid, Pharmacopeia grade castor oil, Ricinoleic acid, Sebacic acid, Sodium undecylenate, Stearic acid, Sulfonated castor oil, Undecanoic acid, Undecylenic acid, Undecylenic alcohol, Undecylenic aldehyde, and Zinc undecylenate.

Sebacic acid and its downstream products are of particular interest. For example, sebacic acid is useful in plasticizers, lubricants, hydraulic fluids, cosmetics, candles, etc. Sebacic acid is also used as an intermediate for aromatics, antiseptics, and painting materials. Accordingly, sebacic acid can be formulated with other components to produce a sebacic acid-containing composition, and/or reacted to form one or more downstream products. Such further products include products chemical compounds, such as esters and polymer resins, including nylon 6/10, which in turn may be fabricated into further products (such as toothbrush bristles, fishing lines, and paper machine felts). Other products include or polymers comprising sebacic acid such as polyester resins, coatings, and adhesives, or the products of reacting sebacic acid with various alcohols to produce sebacate esters, which are useful as plasticizers (which soften stiff plastics and resins) for example in producing polyvinyl chloride (PVC) films to provide low temperature flexibility and freedom from cracking. Alternatively, sebacic acid can be formulated in products such as antifreeze coolants, corrosion inhibitors which can be useful in cutting and metal-working fluids, and in other formulated products such as coatings, and lubricants.

Accordingly, a process for the further formulation and/or reaction of sebacic acid obtained in accordance with the present invention, and the additional products derived therefrom, provides further embodiments of the present invention.

Likewise, a process for the further formulation and/or reaction of 2-octanol obtained in accordance with the present invention, and the additional products derived therefrom, provide further embodiments of the present invention. For example, 2-octoanol may be useful in the preparation of perfumes.

Likewise, a process for the further formulation and/or reaction of biodiesel obtained in accordance with the present invention, and the additional products derived therefrom, provide further embodiments of the present invention.

Likewise, a process for the further formulation and/or reaction of glycerol obtained in accordance with the present invention, and the additional products derived therefrom, provide further embodiments of the present invention. For example, glycerol may be used in the food industry, for inclusion in foods and beverages. It can serve as a humectant, solvent, and/or sweetener, and may help preserve foods. It is also used as filler in commercially prepared low-fat foods (e.g., cookies), and as a thickening agent in liqueurs. As a food additive, glycerol is labelled as E number E422. It can added to icing (frosting) to prevent it setting too hard. Glycerol can be used in pharmaceutical and personal care applications, mainly as a means of improving smoothness, providing lubrication and as a humectant. It is found in allergen immunotherapies, cough syrups, elixirs and expectorants, toothpaste, mouthwashes, skin care products, shaving cream, hair care products, soaps and water-based personal lubricants. In solid dosage forms like tablets, glycerol can used as a tablet holding agent.

Glycerol can be used as a component of glycerin soap, to which essential oils may be added for fragrance. Glycerol can be used as an ingredient in many bath salts recipes. Glycerol can be used as a laxative. It can be taken orally to cause a rapid, temporary decrease in the internal pressure of the eye, which may be a useful initial emergency treatment of severely elevated eye pressure. Glycerol can be formulated in anti-freeze compositions. It can be used as a component of solvents for enzymatic reagents stored at temperatures below 0° C. due to the depression of the freezing temperature. It is also used as a cryoprotectant where the glycerol is dissolved in water to reduce damage by ice crystals to laboratory organisms that are stored in frozen solutions, such as bacteria, nematodes, and mammalian embryos. Glycerol can be used as a chemical intermediate, for example in the production of nitroglycerin, and various explosives such as dynamite, gelignite, and propellants like cordite. Glycerol can be used in hydrogen gas production units, in the production of glycerine acetate (as a potential fuel additive), in the conversion to propylene glycol, in the conversion to acrolein, in the conversion to ethanol, and/or in the conversion to epichlorohydrin (a raw material for epoxy resins).

The skilled person will appreciate that the same principles and process equipment can also be used for other types of reactions with FAEs and/or fatty acids, to reduce both reaction times and also to avoid the use of toxic metal catalysts.

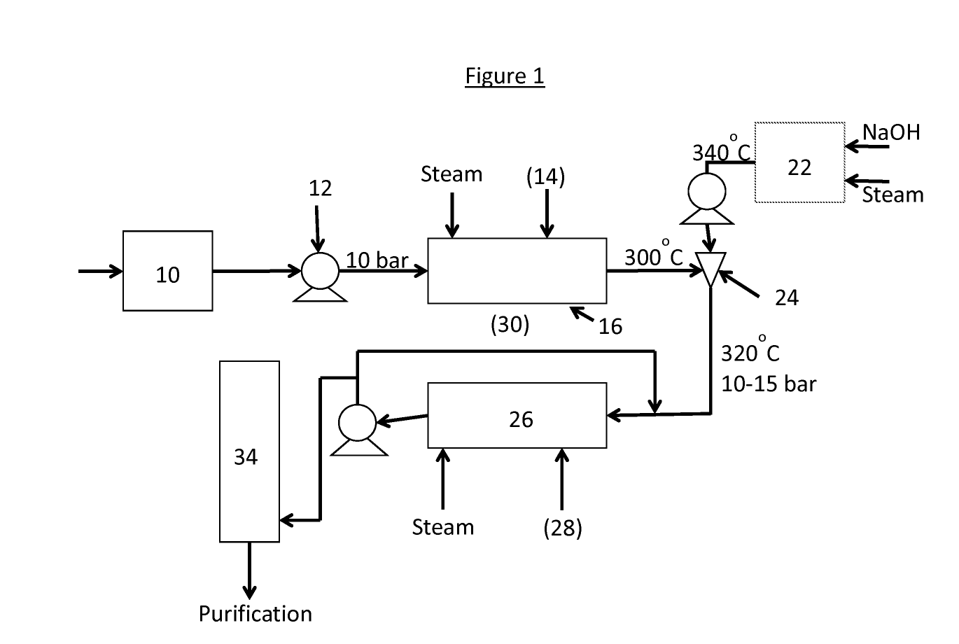

FIG. 1 shows the sequence of the conversion of raw castor oil to sebacic acid according to one preferred embodiment. Castor oil, for example as recovered from plant material by expeller presses, hexane extraction or derived from cell culture, is pumped into a storage tank (10). This oil is preferably filtered to remove the bulk particulates down to 5 microns or smaller in size. A typical castor oil contains about 87% ricinoleic acid which, is the component of the castor oil that converts to sebacic acid.

For the first step oil pre-heating stage, a high pressure oil pump (12), as described above, pushes the stored oil into the lumen of one or more tubes or other conduits of a heat exchanger (14) at a pressure range of from 10 bar to 50 bar, preferably at a pressure of 10 bar. The heat exchanger (14) is usually a shell and tube or plate heat exchanger. The pumped oil is subjected to high pressure steam which is used to heat the oil from 300° C. or up to 320° C., preferably to 300° C. The oil then passes through the lumen of one or more tubes or other conduits of the heat exchanger (14), passing over one or more static mixers (16), which are positioned in series within the lumen(s) to mix the flow of oil and thus ensure that the fluid temperatures are uniform from the inside pipe wall to the center line of the direction of flow of the stream of reaction products.

Concurrently, inorganic base, preferably a 50% sodium hydroxide solution or higher concentration by weight, is pumped from a reserve storage tank (not shown) to a jacketed vessel (22) with hot steam to provide hot sodium hydroxide. The fluid temperature of the hot sodium hydroxide ranges between 300 to 340° C., preferably the temperature is 340° C., and must be pumped under pressure to permit it to be combined with the pressurized hot oil.

For the second step of reacting the oil with the sodium hydroxide, the hot oil and hot sodium hydroxide are pumped using a pump (22) through an injector tee or with parallel piping, through at least one sonic nozzle (24). This generates a lot of noise and requires acoustic baffling to reduce the decibel level below 70 db.

Preferably, at least one sonic nozzle (24) is used to pump the hot sodium hydroxide into the stream of oil as it passes through the lumen of one or more tubes or other conduits of a heat exchanger. However, there can also be two or more sonic nozzles (24) in series. The reaction rates using sonic nozzles (24) are dramatically faster than using laminar flow fluids. When more than one nozzle (24) is used, the temperature gradually increases with each sonic nozzle (24) used because hot sodium hydroxide is injected on the upstream side of the nozzle (24). At this point the caustic fluids and oil may be at a temperature from 300 to 360° C., preferably 320° C. and under a pressure from 10 to 15 bar depending on the concentrations of sodium hydroxide used. Internal pressure is created from the increase in temperature, which converts the water present in the sodium hydroxide solution into steam.

The heated mixture stream passes through the lumen of one or more tubes or other conduits of a heat exchanger (26), typically a shell and tube heat exchanger, with pipe sizes ranging from approximately 1 inch to 2.5 inches in diameter (25.4 mm to 63.5 mm). The lumen of one or more tubes or other conduits of the heat exchanger tubes also have at least one static mixer (28) to maintain a homogeneous temperature throughout the reaction mixture stream.

A recycle loop (30) is used around the heat exchanger (26). The production can be either batch or continuous. Samples of the fluids can be taken regularly and run to determine the rate of conversion of the reactants into products, for example the conversion of ricinoleic acid into sebacic acid.

When the reaction is, for example, at least 70% converted from starting materials into products, a recirculation pump (32) used for the recycle loop (30) is used to pump the reaction mixture stream to a distillation column (34), in order to begin the third step of product separation. This column can be atmospheric pressure or partial vacuum.

After distillation, various byproducts are separated from the sebacic acid including 2-octanol. In some embodiments of the invention, the reaction mixture stream is fed from the recycle loop to a settling tank (36) in order to first separate the aqueous fractions by gravity.

The unreacted sodium hydroxide can also be recovered from the recovered products by using ion exchange resins, or, alternatively can be neutralized using some dilute acid.

Any darkening of the oil fraction or the sebacic acid can be removed using techniques well-known to the skilled person such as nanofiltration or by using activated charcoal.

The obtained sebacic acid can then be purified in several ways depending on the end product quality requirements. Any unreacted castor oil or ricinoleic acid is recycled back to the beginning of the process.

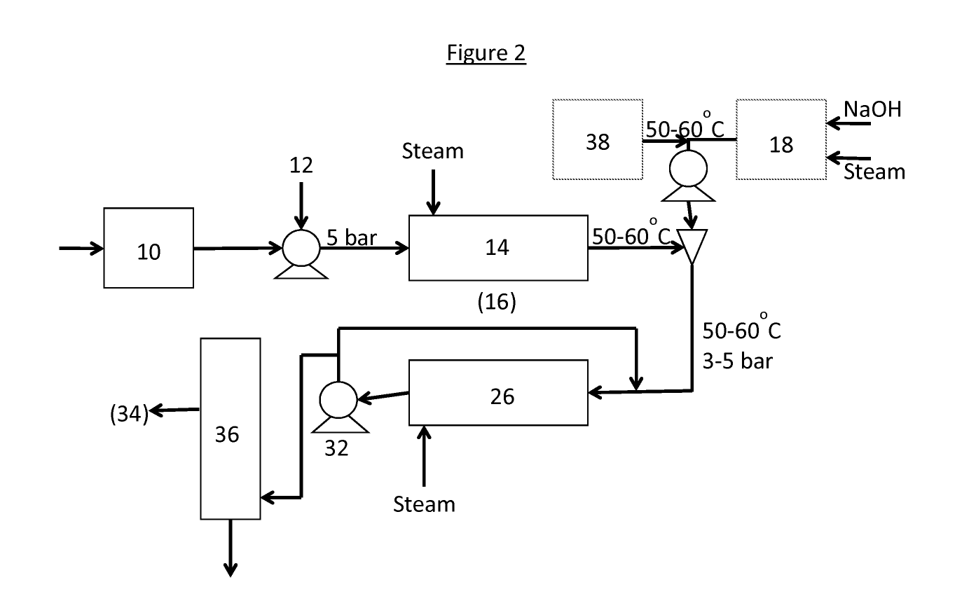

FIG. 2 shows the sequence of the synthesis of biodiesel from rapeseed or canola oil.

The synthesis of biodiesel is very similar to the process described above already for castor oil.

The main differences are:
methanol or ethanol is also added for the transesterification step to produce glycerin.
the unreacted oil, glycerin and product biodiesel can be separated by using a settling tank (36).
the main purpose of the distillation column (34) is to remove and recover the methanol which has a high vapor pressure. The waste glycerin can be purified as a separate product.

The reaction temperatures in the synthesis of biodiesel are also different. The high pressure oil pump (12), operates at a pressure range of from 5 to 10 bar, preferably at a pressure of 5 bar.

The inorganic base, preferably a 50% sodium hydroxide solution or higher concentration by weight, is pumped from a reserve storage tank (not shown) to a jacketed vessel (18) with hot steam to provide hot sodium hydroxide. Concurrently, methanol or ethanol is pumped from a storage tank, and mixed with the hot sodium hydroxide. The fluid temperature of the hot inorganic base and alcohol mixture ranges between 50 to 60° C.

The use of methanol and the sonic nozzles dramatically lowers the reaction temperatures compared to sebacic acid. Therefore the step where the at least one sonic nozzle (24) pumps the inorganic base/alcohol mixture into the reaction stream is performed at a temperature of about 50 to 60° C.

Advantageously, the correspondingly lowered vapor pressures and operating process pressures result in the equipment required to be less expensive than the sebacic acid embodiment. Compared to traditional process methods to synthesize biodiesel, the present invention allows for much faster reaction times, going from 3 hours down to less than 15 minutes for a batch run. This provides much higher throughput capacity in the plant in the same capital equipment.

The invention will be further understood with reference to the following non-limiting experimental examples.

### Comparative Examples

Sebacic acid is manufactured according to a preferred protocol of the present invention as described above for FIG. 1. A typical reaction, without the inclusion of any catalyst, consists of:

298 g castor oil fatty acid (1.0 moles)
120 g sodium hydroxide (3.0 moles)
105 g water (5.8 moles)

The temperature of the second step reaction is 320° C. at the point of combining oil and sodium hydroxide, and the reaction mixture is held at temperature above 250° C. for 57 minutes as it passes through the heat exchanger tubes under continuous flow. The reaction yields 128.5 g sebacic acid, which is a 74.6% yield. Importantly, the reaction product contains no toxic catalyst.

In comparison, using a typical reaction with catalyst as known in the art, the reaction consists of:

298 g castor oil fatty acid (1.0 moles)
120 g sodium hydroxide (3.0 moles)
105 g water (5.8 moles)
4.95 g lead chloride
0.9 g cadmium oxide

The temperature of the reaction is 320° C., time reaction performed above 250° C. is for 58 minutes, yield is 139.5 g sebacic acid which is 81.2% of the theoretical yield.

Depending on the ratios of castor oil, catalyst, sodium hydroxide and temperature the yields of sebacic acid with catalysts range from 70% to 85%. The yields of sebacic acid without catalysts range about 65% to 75%. The above reactions times are for static conditions which means that they are sealed within a high pressure vessel without simple mixing.

Despite the reduced reaction yield, the ability to efficiently generate sebacic acid on an industrial scale without the use of toxic catalyst is highly important development, which opens new opportunities and flexibility to manufacture sebacic acid whilst complying with regulatory requirements for environmental compatibility, which is not possible with processes that use toxic catalyst.

1. A process for the reaction of a fatty acid ester and/or a fatty acid with an inorganic base, the process comprising the following steps:
(i) providing a pressurised and heated stream of fatty acid ester and/or fatty acid;
(ii) providing an inorganic base;
(iii) combining the stream obtained from step (i) with the inorganic base obtained in step (ii), in the presence of water, in a reaction vessel, by injection, thereby to produce reaction mixture in the form of a pressurised and heated stream;
(iv) optionally maintaining the stream obtained in step (iii) at a selected temperature and pressure; and
(v) thereby obtaining a reaction product.

2. The process of claim 1 which is conducted in the absence of transition metal catalyst.

3-23. (canceled)

24. Use of one or more static mixers, optionally made of stainless steel, to mix a heated and pressurised stream of castor oil, wherein the stream passes through the lumen of a tube or other conduit, and the one or more static mixers are positioned within the lumen.

25. (canceled)

26. Use of one or more sonic nozzles to introduce inorganic base into a reaction vessel, thereby combining the inorganic base with fatty acid ester and/or fatty acid.

27. The use of claim 26, for performing the process according to claim 1.

28-35. (canceled)

36. The process of claim 1, wherein step (iii) comprises combining the stream obtained from step (i) with the inorganic base obtained in step (ii), in the presence of water and a C₁-C₃ mono-alcohol (such as methanol, ethanol or propanol), in a reaction vessel, by injection, thereby to produce reaction mixture in the form of a pressurised and heated stream.

37. The process of claim 1, wherein step (i) comprises providing a pressurised and heated stream of fatty acid ester, wherein at 25° C. and 1 atm the fatty acid ester has a viscosity of less than about 200 cP, about 150 cP, or about 100 cP.

38. The process of claim 36, wherein the fatty acid ester comprises canola or rapeseed oil, and preferably the reaction product comprises biodiesel and/or glycerol.

39. The process of claim 38 wherein the biodiesel is, or comprises, or consists essentially of, or consists of, one or more compounds selected from fatty acid methyl ester, fatty acid ethyl ester, and/or fatty acid propyl ester.

40. The process of claim 38, wherein the fatty acid ester comprises canola or rapeseed oil, and step (i) of claim comprises pumping canola or rapeseed oil at a pressure of about 5 bar 10, and at a temperature from about 50° C. to about 80° C.

41. The process according to claim 36, wherein step (i) of claim 1 comprises steam injection into the stream of pumped fatty acid ester.

42. The process of claim 36, wherein the inorganic base is sodium hydroxide or potassium hydroxide.

43. The process of claim 36, wherein the inorganic base is injected into the heated and pressurised stream of fatty acid ester and/or fatty acid using one or more sonic nozzles or, in an alternative, the inorganic base is blended with fatty acid ester and/or fatty acid and the blend is passed through one or more sonic nozzles.

44. The process of claim 36, wherein the fatty acid ester is canola or rapeseed oil and it is reacted with the inorganic base, in the presence of water and C₁-C₃ mono-alcohol (such as methanol or ethanol), at about 50-60° C. under pressure of about 3-5 bar.

45. The process of claim 36, wherein the fatty acid ester and/or fatty acid is reacted with inorganic base, in the presence of water and C₁-C₃ mono-alcohol (such as methanol or ethanol), in a continuous unidirectionally flowing stream, wherein the stream passes over and is mixed by one or more in-line static mixers.

46. The process of claim 36, wherein the fatty acid ester and/or fatty acid is reacted with inorganic base, in the presence of water and C₁-C₃ mono-alcohol (such as methanol or ethanol), in the lumen of one or more tubes or conduits, and preferably the one or more tubes or conduits form a heat exchanger.

47-58. (canceled)

59. The process of claim 1, wherein the reaction products produced following step (v) comprise no, or substantially no, transition metal catalyst, and preferably wherein the reaction products comprise sebacic acid and/or 2-octanol.

60. The process of claim 1, wherein the reaction products produced following step (v) comprise no, or substantially no (such as less than 10 by weight), non-reactive diluents or other non-reactive processing aids which reduce the viscosity of the reaction mixture, including mineral oil, polyethylene glycol (PEG), waxes and/or paraffin, and preferably wherein the reaction products comprise sebacic acid and/or 2-octanol.

61. The process of claim 1, wherein following step (v), one or more of the reaction products is/are collected and/or purified, preferably wherein the collected and/or purified products are selected from the group consisting of sebacic acid, 2-octanol, biodiesel, and glycerol.

62. The process of claim 61 wherein the collection and/or purification comprises feeding the reaction product collected from step (v) into a settling tank.

63-87. (canceled)
